(19) United States
(12) Patent Application Publication
(10) Pub. No.: US 2024/0011166 A1
(43) Pub. Date: Jan. 11, 2024

(54) SUSTAINABLE BIOMASS PRODUCTION

(71) Applicant: DSM IP ASSETS B.V., Heerlen (NL)

(72) Inventors: Hendrik Jan NOORMAN, Echt (NL); Reinier Franciscus Petrus GRIMBERGEN, Echt (NL)

(21) Appl. No.: 18/252,156

(22) PCT Filed: Nov. 9, 2021
(86) PCT No.: PCT/EP2021/081069

§ 371 Date: May 8, 2023

(30) Foreign Application Priority Data
Nov. 10, 2020 (EP) 20206739.3

(51) Int. Cl. C25B 3/26; C25B 3/07

(52) U.S. Cl. CPC C25B 3/26; C25B 3/07

(57) ABSTRACT

The invention is directed to a method for the production of biomass comprising the steps of (i) capturing CO₂ from a CO₂ containing gas stream, and subsequently reducing the captured CO₂ to a reduced CO₂ product; and (ii) an anaerobic fermentation, wherein a carbon substrate is used for the production of an organic feedstock; and (iii) an aerobic fermentation, wherein the organic feedstock is used for the production of biomass; wherein the reduced CO₂ product is fed to the anaerobic fermentation and/or to the aerobic fermentation and the co-produced oxygen is fed to the aerobic fermentation.

### FIELD OF THE INVENTION

The invention is directed to a method for the production of biomass, in particular single cell protein.

### BACKGROUND OF THE INVENTION

Single cell protein (SCP) refers to microbial biomass that can be used in protein-rich human and animal feeds. SCP can replace conventional sources of protein supplementation such as soymeal or fishmeal.

Attempts are being made in industry to produce organic chemicals from waste materials, using renewable energy and renewable feedstocks. It is a challenge to provide new pathways to enable the production of a wide variety of chemicals.

WO 2016/187494 describes a method for producing an animal feed by culturing (e.g. anaerobic) microorganisms to produce microbial biomass. The animal feed is produced by fermentation of a gaseous substrate. The substrate refers to a carbon and/or energy source for the microorganisms. The substrate may be derived from a waste or off-gas obtained as a byproduct of an industrial process. The substrate preferably comprises about 15-70 mol % CO. Examples of suitable substrate mentioned in WO 2016/187494 are steel mill or blast furnace gas, basic oxygen furnace gas and syngas. Accordingly, it is known from WO 2016/187494 to use an off-gas from steel industry (e.g. basic oxygen furnace (BOF) gas) as a carbon and/or energy source for the microorganisms producing SCP.

A disadvantage of WO 2016/187494 is that a substantial part of the off-gas still leaves the process as waste. For example, the CO₂ content in industrial off-gases can be high. Further, part 2 of the CO is converted in additional CO₂. However, WO 2016/187494 does not provide a purpose for the CO₂ present in the off-gas.

EP3715464A1 relates to a method for cultivating a microorganism capable of utilizing an organic feedstock, comprising cultivating a microorganism in one or more bioreactors, capturing CO₂ from the one or more bioreactors in a capturing unit and reducing the CO to an organic feedstock, for instance in a reduction unit and feeding at least part of the organic feedstock into the one or more bioreactors. In the method of EP3715464A1 water is electrolysed into H₂ and O₂ in a separate electrolysis unit requiring a high amount of electricity and the produced H₂ is used to reduce CO₂ in another separate reactor at high temperature. A disadvantage of this two-step process for reducing CO₂ is that it is energy and capital intensive. A further disadvantage of the method disclosed in EP3715464A1 is that sugar is still used in the cultivation of the microorganism and at least part of CO₂ produced in the aerobic fermentation leaves the system.

WO2016/070160 discloses a method for the production of biomass or lipids by feeding a gaseous substrate to an anaerobic fermenter to produce an acid or alcohol product (e.g. acetate). The gaseous substrate may be a CO or CO₂-containing waste gas obtained as a by-product of an industrial process. The acid or alcohol product is fed into an aerobic fermenter wherein lipids and non-lipid biomass are produced by microalgae. A disadvantage of the method disclosed in WO2016/070160 is that CO₂ produced in fermentation still leaves the fermentation system.

### BRIEF DESCRIPTION OF THE DRAWINGS

FIG. 1 shows a schematic representation of one embodiment of the method of the invention. A first waste gas containing CO and CO₂ is fed to an anaerobic fermentation. In the anaerobic fermentation, an organic feedstock is produced using CO from the waste gas as a carbon substrate. The off-gas from the anaerobic fermentation contains CO₂, which is captured and subsequently electrochemically reduced, forming a reduced CO₂ product and O₂. The reduced CO₂ product is a carbon substrate that is fed to the anaerobic fermentation and aerobic fermentation. In the aerobic fermentation, the reduced CO₂ product is used as a carbon substrate, and the organic feedstock is converted into biomass. The off-gas from the aerobic fermentation contains CO₂ and is fed to the carbon capture. Furthermore, CO₂ is also captured from a second waste gas.

### SUMMARY OF THE INVENTION

It is an object of the invention to provide a more sustainable route to prepare (microbial) biomass or SCP from industrial off-gases.

In particular, it is an object to provide a method for preparing (microbial) biomass wherein the net amount of waste produced is zero.

More in particular, it is an object to provide a method for preparing (microbial) biomass, wherein all byproducts formed are used to improve the (microbial) biomass production.

At least one of these objects has been achieved by providing a method for the production of biomass comprising the steps of (i) capturing CO₂ from a CO₂ containing gas stream, and reducing the captured CO₂ via electrochemical reduction to a reduced CO₂ product and producing a O₂ stream; and (ii) an anaerobic fermentation, wherein a carbon substrate is used for the production of an organic feedstock; and (iii) an aerobic fermentation, wherein the organic feedstock is used for the production of biomass; and wherein the reduced CO₂ product is fed to the anaerobic fermentation and/or to the aerobic fermentation.

### DETAILED DESCRIPTION OF THE INVENTION

In a first aspect, the invention provides a method for the production of biomass comprising the steps of:
(i) capturing CO₂ from a CO₂ containing gas stream, and subsequently reducing the captured CO₂ via electrochemical reduction to a reduced CO₂ product and producing a O₂ stream; and
(ii) an anaerobic fermentation, wherein a carbon substrate is used for the production of an organic feedstock; and
(iii) an aerobic fermentation, wherein the organic feedstock is used for the production of biomass; and wherein the reduced CO₂ product is fed to the anaerobic fermentation or to the aerobic fermentation.

By capturing and converting CO₂, a carbon product is obtained that may improve the (carbon) efficiency of the anaerobic or aerobic fermentation. For example, the reduced CO₂ product can be used in the aerobic and/or anaerobic fermentation as a carbon substrate, in particular as carbon or energy source for the microorganisms. Moreover, the combination of CO₂ capture and subsequent CO₂ reduction allows for a process wherein a waste gas can provide a substantial part of the carbon source and/or energy source for the fermentation.

Preferably, at least part of the captured CO₂ and/or at least part of the carbon substrate used in the anaerobic fermentation originates from a waste gas. Accordingly, a waste gas may be fed to the anaerobic fermentation or to the CO₂ capture or to both. The waste gas fed to the anaerobic fermentation may be the same or a different gas from the waste gas fed to the CO₂ capture. The waste gas preferably comprises both a gaseous carbon substrate (in particular CO) and CO₂. In a preferred embodiment, the anaerobic fermentation removes at least part of the CO from the waste gas, and the CO₂ capture removes at least part of the CO₂ from the waste gas. The order in which CO and CO₂ are removed from the waste gas is not particularly critical. However, it is preferred that first CO is removed, and subsequently CO₂. The advantage of first removing CO is that CO can be used directly in the fermentation and CO₂ needs to be reduced first. Removal of CO before the reduction step prevents the formation of formaldehyde which is toxic in the fermentation.

In one embodiment the carbon substrate in step (ii) of the method for producing biomass as disclosed herein, comprises the reduced CO₂ product, a gaseous carbon substrate, preferably CO, from waste gas, or a mixture of a reduced CO₂ product and a gaseous substrate from waste gas. The waste gas may be an industrial waste gas, such as an off-gas from steel industry.

Preferably, the waste gas is selected from basic oxygen furnace (BOF) gas, blast furnace (BF) gas, coke oven gas (COG), and mixtures of two or more of these gases. The waste gas may comprise Hz, for example in an amount of 1-10%. The H₂ present in the gas may have a positive effect on the fermentation steps. H₂ is for example present in BF gas. The waste gas may comprise significant amounts of CO, for example in the range of 5-75 vol. %. BOF gas typically contains vol. % CO, while BF gas typically contains 15-25 vol. % CO. The CO present in the gas may have a positive effect on the fermentation steps. The waste gas may further comprise nitrogen gas (N₂). Alternatively, the waste gas may be an industrial waste gas from energy-intensive industrial processes such as fertilizer industry (e.g. from the Haber process), a waste gas obtained from Steam Methane Reforming (SMR) and/or AutoThermal Reforming (ATR), a waste gas obtained in TiO₂ production, or a waste gas obtained in cement production.

An advantage of the present process having two separate fermentation steps and electrochemical reduction of CO₂ is that an improved process is provided, wherein CO₂ produced and fed to the system is fully used in and recycled to the anaerobic and aerobic fermentation, and the efficiency of biomass production is increased. In addition a little amount of or no H₂ is formed during electrochemical reduction of CO₂ and much less energy is needed for the reduction CO₂ as compared to the method disclosed in EP3715464A1. The present anaerobic and aerobic steps have a very high yield, fast pace and are safe operations. In single processes, such as disclosed in WO 2016/187494, safety issues like knallgas (oxyhydrogen) or explosive mixtures of CO and O₂ require expensive measures. Further, gas dilution results in low mass transfer. In one step anaerobic processes, such as disclosed in WO 2016/187494, the conversion yield of CO/H₂ to protein and the reaction rate are relatively low. Another advantage of the method of the present invention is that less or no sugar, such as sucrose or glucose, is used in the anaerobic and aerobic fermentation compared to a process that uses wherein sugar is used, such as disclosed in EP3715464A1, which reduces land use and further reduces CO₂ emissions,

In one embodiment in the method according to the present invention, no sugar is added to the anaerobic fermentation (step ii) and aerobic fermentation (step iii).

In step (i) of the method of the invention, CO₂ is captured from a CO₂ containing gas stream. This step (i) may also be referred to as the carbon capture or CO₂ capture. After CO₂ capture, the captured CO₂ is reduced via electrochemical reduction, resulting in a reduced CO₂ product. The reduced CO₂ product typically comprises a C1 compound, i.e. a compound with one carbon atom. The reduced CO₂ product is preferably selected from formic acid, carbon monoxide, methanol and formaldehyde. The reduced CO₂ product may for example contain at least 25 wt. %, at least 50 wt. % or at least 75 wt. % of said C1 compound.

The CO₂ containing gas stream is preferably a gas stream comprising at least 1 vol. %, more preferably at least 5 vol. %, even more preferably at least 10 vol. %, most preferably at least 15 vol. % CO₂. Such high CO₂ concentrations in the gas stream provide for an easier and/or more efficient CO₂ capture.

The CO₂ containing gas stream is preferably a waste gas. For example, the CO₂ containing gas stream may be a stream of the waste gas described above (such as BOF, BF or COG gas).

The CO₂ containing gas stream may also be an off-gas from the anaerobic fermentation and/or from the aerobic fermentation. The CO₂ containing gas stream may also be a mixture of any of the aforementioned waste and off-gases.

In a preferred embodiment, step (i) comprises capturing CO₂ from off-gas from the anaerobic fermentation and/or from the aerobic fermentation. In this case, at least part of the CO₂ containing gas stream will comprise a waste stream from the anaerobic fermentation and/or a waste stream from the aerobic fermentation. This has the advantage that carbon containing waste from the fermentation can be re-introduced as a carbon or energy source in the fermentation steps. Such an off-gas may comprise considerable amounts of CO₂, in particular when a CO₂ containing waste gas from anaerobic fermentation is used. Advantageously, the off-gas from the anaerobic fermentation comprises at least 90 vol. % CO₂, preferably at least 95 vol. % CO₂. CO₂ present in the off-gas may at least in part originate from waste gas fed to the anaerobic fermentation.

CO₂ may be captured from the CO₂ containing gas stream by any suitable technique known in the art. For example, CO₂ can be captured by separating CO₂ from the CO₂ containing gas stream using one or more of absorption, adsorption, and membrane gas separation.

Typically, CO₂ capture is conducted using a capture solvent. The CO₂ containing gas stream is contacted with the capture solvent, thereby absorbing CO₂ from the gas stream to form a CO₂ rich capture solvent. The capture solvent will capture CO₂ from the gas stream by absorbing the CO₂ in the solvent.

The capture solvent may comprise a physical solvent and/or a chemical solvent. The solvent may be an aqueous or non-aqueous solvent. The solvent may comprise one or more selected from the group consisting of dimethyl ethers of polyethylene glycol, M-methyl-2-pyrrolidone, methanol, and propylene carbonate. The physical solvent may for example be a mixture of various dimethyl ethers of polyethylene glycol. The chemical solvent may be a solution of an amine or a salt in a solvent, e.g. in water. The chemical solvent is preferably an amine-based solvent. Suitable chemical solvents are aqueous solvents, for example aqueous solutions of one or more compounds selected from 2-amino-2-methyl-1-propanol (AMP), tertiary amine (e.g. MDEA), methyl diethanolamine, and ammonia. Such species form bicarbonates upon loading with CO₂. Other suitable chemical solvents are aqueous solutions comprising ethanolamine (e.g. monoethanolamine, N-methyl diethanolamine or diglycolamine). Further suitable chemical solvents are aqueous solutions of inorganic salts, such as e.g. aqueous solutions of KOH, NaOH and NH₄OH.

After absorption, CO₂ may be released and/or separated from the capture solvent. CO₂ may be transferred from the capture solvent to a gas or liquid stream that is more suitable for the reduction reaction.

Preferably, CO₂ is reduced via electrochemical reduction, thereby producing the reduced CO₂ product. In this case, CO₂ capture may be followed by the steps of CO₂ release, purification, and recovery of the carbon capture solvent, solubilizing CO₂ in an (aqueous, organic or inorganic) electrolyte, and electrochemically converting the solubilized CO₂ to the CO₂ reduced product. However, for a more efficient process, a so-called integrated process, an electrochemical cell can be used to directly react solubilized CO₂ from the capture solvent. The CO₂ release and purification steps may thus be omitted.

Preferably, no H₂ is formed in step (i) of reducing CO₂ to a reduced CO₂ product.

CO₂ reduction may be conducted at elevated temperature and optionally also increased pressure. High temperatures may be advantageously used to increase the efficiency of the electrochemical reduction. The temperature during electrochemical reduction may be in the range from 20 to 100° C., preferably 30-80° C., more preferably 50-80° C. Electrochemical reduction may be conducted at a pressure of more than 1 bar, preferably in the range of 2-20 bar, such as 5-10 bar. An example wherein increased pressure and/or elevated temperature are used is an embodiment wherein electrochemical reduction is conducted in a solid oxide electrolysis cell.

For the electrochemical reduction of CO₂, the captured CO₂ may be reduced at the cathode of an electrochemical cell. This can be achieved by introducing the CO₂-rich capture solvent into a cathode compartment of the electrochemical cell; and applying an electrical potential between an anode and a cathode in the electrochemical cell sufficient for the cathode to reduce the CO₂ into the reduced CO₂ product. Reduction thus takes place in the CO₂-rich capture solvent, and results in a CO₂-poor capture solvent. The CO₂ reduced product is collected and fed to the anaerobic or aerobic fermentation.

The reduced CO₂ product may comprise one or more components selected from the group consisting of alkanes, alkenes, carbon monoxide, carboxylic acids, alcohols, aldehydes, and ketones. More specifically, the reduced CO₂ product may comprise one or more components selected from the group consisting of carbon monoxide, methane, ethane, ethylene, methanol, ethanol, formaldehyde, acetaldehyde, 1-propanol, formic acid, oxalic acid, glyoxylic acid, glycolic acid, acetic acid, tartaric acid, malonic acid, propionic acid, and salts thereof. Preferably, the reduced CO₂ product is a C1 compound, more preferably formic acid, carbon monoxide, methanol or formaldehyde. These C1 compounds can be efficiently used as carbon or energy source for the anaerobic and/or aerobic fermentation.

By controlling the electrical potential between the anode and the cathode, and/or by selecting a suitable cathode catalyst or suitable catholyte composition in the electrochemical cell, the desired product or products may be obtained.

Additionally, H₂ can be a byproduct at the cathode side. Usually the amount of H₂ formed is small. This can be advantageously used in fermentation to contribute to the conversion. Preferably, the present method does not comprise a separation step for purifying the reduced CO₂ products, such as to separate CO from H₂.

At the anode side of the electrochemical cell, preferably a product is generated that can be suitably used in the method of the invention. Preferably, oxygen (O₂) is generated at the anode of the electrochemical cell. Accordingly, the electrochemical reduction results in a reduced CO₂ product and an O₂ stream. The method of the invention may in such case further comprise the step of feeding the obtained O₂ stream to the aerobic fermentation. An O₂ stream produced in an electrochemical cell typically has a very high purity, for example more than 95 vol. % O₂, or even more than 99 vol. % O₂. An O₂ stream with such high purity can be used in the aerobic fermentation to improve the fermentation process.

A known procedure for capturing and electrochemically reducing CO₂ is for example described in WO 2019/160413 and WO 2019/172750.

The method according to the invention further comprises the step of feeding at least part of the reduced CO₂ product to the anaerobic fermentation, to the aerobic fermentation, or to both.

Depending on the type of reduced CO₂ product, it may be suitably fed to the anaerobic fermentation, to the aerobic fermentation, or both.

Formic acid can be used as an energy source for microorganisms. Accordingly, when the reduced CO₂ product is formic acid, it can be suitably fed to one or both of the anaerobic and aerobic fermentation. The formic acid may be fed in the form of a salt, for example as ammonium, calcium, magnesium, potassium, or sodium salt.

CO and methanol can be used by the microorganisms as a reactant in producing the organic feedstock. Accordingly, when the reduced CO₂ product is CO or methanol, it can be suitably fed to the anaerobic fermentation.

Formaldehyde may be used as a carbon source in fermentation, but is preferably only added to the fermentation in relatively low concentrations in view of toxicity. Formaldehyde can for this purpose also be fed as a formaldehyde derivative. Such a derivative may have lower toxicity than formaldehyde, in particular towards the microorganisms in the fermentation. The derivative may be selected from trioxane, paraformaldehyde and methane-diol. Preferred derivatives are trioxane and paraformaldehyde. In case formaldehyde derivatives are fed to a fermentation step, the method of the invention may comprise the additional step of converting formaldehyde to a derivative that has lower toxicity towards the microorganisms in the fermentation.

Step (ii) of the method of the invention is an anaerobic fermentation for the production of an organic feedstock. A carbon substrate is used for the production of the organic feedstock. The carbon substrate may be a C1 source. The C1 source may e.g. be used as energy source by microorganisms or for the production of the reduced CO₂ product. The reduced CO₂ product is preferably fed to the aerobic fermentation and may be used as C1 source.

In one embodiment, the anaerobic fermentation in step ii) in the method according to the present invention further comprises feeding a CO containing waste gas to the anaerobic fermentation, wherein CO is used as a C1 source or a carbon substrate for the fermentation.

The CO containing waste gas preferably further contains CO₂. CO₂ present in the waste gas can be captured in the CO₂ capture in step (i). The waste gas may first be fed to the anaerobic fermentation before capturing CO₂ from the waste gas. However, it is also possible to first subject the CO containing waste gas to CO₂ capture before feeding it to the anaerobic fermentation.

The CO containing waste gas may further comprise nitrogen and/or hydrogen.

The CO containing waste gas may be a waste gas as defined above. Accordingly, it may be an off-gas from steel industry, preferably selected from basic oxygen furnace (BOF) gas, blast furnace (BF) gas, coke oven gas (COG), or mixtures thereof.

Before feeding the CO containing waste gas to the anaerobic fermentation, a washing step may be conducted to remove toxic compounds, such as hydrogen cyanide. Washing may be performed using a scrubber.

The organic feedstock produced in step (ii) may be chosen from the group consisting of acetate, acetic acid, ethanol, butanol, acetone, butyrate, isopropanol, or mixtures thereof. Preferably, the organic feedstock is acetic acid, ethanol, butanol, acetone or isopropanol or mixtures thereof. A disadvantage of organic feedstocks like formate, acetate, or butyrate is that these compounds are anions which need to be balanced with a cation, for instance added through titration. In a subsequent fermentation the uptake of the acid results in the need for back-titration to balance the cation resulting in extra salt being produced as a by-product.

Preferably the present step (ii) of an anaerobic fermentation, wherein a carbon substrate is used for the production of an organic feedstock, comprises cultivating a microorganism belonging to the genus Clostridium, Cupravidus, Moorella and Sporomusa, preferably wherein the microorganism produces the organic feedstock and/or wherein the microorganism utilizes the carbon substrate.

Preferably the present step (ii) of an anaerobic fermentation, wherein a carbon substrate is used for the production of an organic feedstock, comprises cultivating a microorganism chosen from the group consisting of Clostridium ljungdahlii, Clostridium acetobutylicum, Clostridium carboxidivorans, Clostridium aceticum, Clostridium autoethanogenum, Clostridium ragsdalei, Clostridium coskatii, Clostridium drakei, Clostridium formicoaceticum, Clostridium magnum, Clostridium scatologenes, Cupriavidus necator, Scenedesmus obliquus, Acetobacterium woodii, C. pyrenoidosa, Sporomusa ovata, Alkalibaculum bacchii, Blautica producta, Butyribacterium methylotrophicum, Eubacterium limosum, Moorella thermautotrophica, Moorella thermoacetica, Oxobacter pfennigii, Sporomusa ovata, Sporomusa silvacetica, Sporomusa sphaeroides, and Thermoanaerobacter kiuvi.

Step (iii) of the method of the invention is an aerobic fermentation for the production of biomass. The organic feedstock obtained in step (ii) is fed to the aerobic fermentation. Further, the reduced CO₂ product may be fed to the aerobic fermentation. The reduced CO₂ product may be used as a C1 source in the aerobic fermentation, e.g. as carbon or energy source by microorganisms.

An aerobic fermentation in step (iii) of the method for producing a biomass comprises cultivating a microorganism, for instance a microorganism such as bacteria, yeast, filamentous fungi or algae. The microorganism in the aerobic fermentation uses the organic feedstock for the production of biomass.

The biomass comprises a microbial biomass, single cell protein or microbial protein. Preferably, the biomass comprises single cell protein, or microbial protein. Single cell protein or microbial protein refers to a protein extracted from microorganisms or a microbial culture.

The biomass comprises biomass from the aerobic fermentation, or comprises biomass from the aerobic and anaerobic fermentation

The microorganisms in the anaerobic and/or aerobic fermentation may be selected from algae, yeast, filamentous fungi and bacteria. The microorganisms may be a yeast such as Saccharomyces cerevisiae, Pichia pastoris, Komagataella pastoris, Komagataella phaffi, Komagataella pseudopastoris, Kluyveromyces lactis, Yarrowia lipolytica, Hansenula polymorpha, Geotrichum candidum, or Candida utilis. The microorganism may also be a filamentous fungi selected from Acremonium, Agaricus, Aspergillus, Aureobasidium, Chrysosporium, Coprinus Filibasidium, Fusarium, Humicola, Magnaporthe, Mucor, Myceliophthora, Neocallimastix, Neurospora, Paecilomyces, Penicillium, Piromyces, Panerochaete, Pleurotus, Schizophyllum, Talaromyces, Rasamsonia, Thermoascus, Thielavia, Tolypocladium, and Trichoderma>Preferably, a filamentous fungus is Penicillium chrysogenum, Aspergillus niger, Acremonium alabamense, Aspergillus awamori, Aspergillus foetidus, Aspergillus sojae, Aspergillus fumigatus, Talaromyces emersonii, Rasamsonia emersonii, Aspergillus oryzae, Chrysosporium lucknowense, Fusarium oxysporum, Myceliophthora thermophila, Trichoderma reesei and Thielavia terrestris.

The present algae are preferably chosen from the group consisting of glaucophytes, rhodoplasts and chloroplasts. Preferably the algae are chosen from the group consisting of glaucophytes, rhodoplasts and chloroplasts. More preferably the present algae are heterotrophic algae, more preferably heterotrophic algae like Chlorella, Nannochloropsys, Nitzschia, Thraustochytrium or Schizochyttrium.

The term “bacteria” includes both Gram-negative and Gram-positive microorganisms. Suitable bacteria may be selected from e.g. Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus, Streptomyces, Actinomycetes, Xanthomonas or Sphingomonas. Preferably, the bacterial cell is selected from the group consisting of B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Rhodobacter capsulatus, Pseudomonas zeaxanthinifaciens, Paracoccus denitrificans, E. coli, C. glutamicum, Staphylococcus carnosus, Streptomyces lividans, Sinorhizobium melioti and Rhizobium radiobacter.

The method for producing biomass may further comprise a step of recovering the biomass from the aerobic fermentation by suitable methods known in the art. Recovering biomass may comprise centrifugation or filtration.

### EXAMPLES

### Example 1

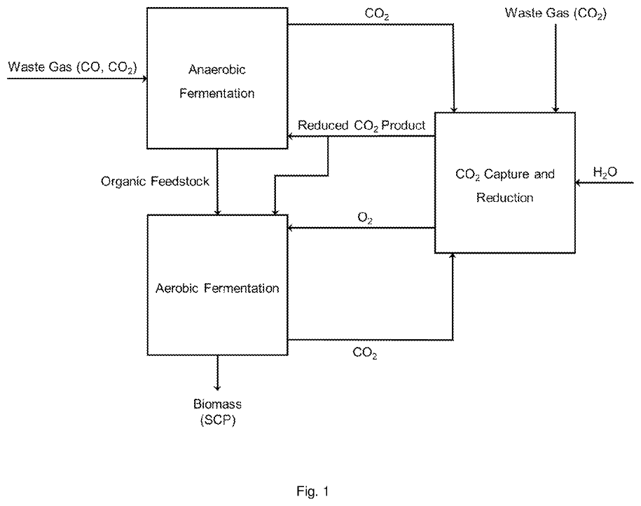

A schematic representation of an embodiment of the invention is given in FIG. 1. A first waste gas containing CO and CO₂ is fed to an anaerobic fermentation. In the anaerobic fermentation, an organic feedstock is produced using CO from the waste gas as a carbon substrate. The off-gas from the anaerobic fermentation contains CO₂, which is captured and subsequently electrochemically reduced, forming a reduced CO₂ product and O₂. The reduced CO₂ product is fed to the anaerobic fermentation and aerobic fermentation. In the aerobic fermentation, the reduced CO₂ product is used as a substrate, and the organic feedstock is converted into biomass. The off-gas from the aerobic fermentation contains CO₂ and is fed to the carbon capture. Furthermore, CO₂ is also captured from a second waste gas.

1. A method for production of biomass comprising:
i. capturing CO₂ from a CO₂ containing gas stream, and subsequently reducing captured CO₂ via electrochemical reduction to a reduced CO₂ product and producing an O₂ stream; and
ii. an anaerobic fermentation, wherein a carbon substrate is used for production of an organic feedstock; and
iii. an aerobic fermentation, wherein the organic feedstock is used for production of biomass; and
wherein the reduced CO₂ product is fed to the anaerobic fermentation and/or to the aerobic fermentation.

2. The method according to claim 1 wherein the biomass comprises single cell protein.

3. The method according to claim 1, wherein the aerobic fermentation comprises cultivating a microorganism for production of biomass.

4. The method according to claim 3, wherein the microorganism is a bacterium, a yeast, a filamentous fungus, or an algae

5. The method according to claim 1, wherein the organic feedstock is chosen from the group consisting of acetic acid, ethanol, butanol, acetone and isopropanol, and mixtures thereof.

6. The method according to claim 1, wherein no sugar is fed to the anaerobic and aerobic fermentation.

7. The method according to claim 1, wherein in i), no H₂ is produced.

8. The method according to claim 1, further comprising feeding the O₂ stream to the aerobic fermentation.

9. The method according to claim 1, wherein (i) comprises capturing CO₂ from off-gas from the anaerobic fermentation and/or from the aerobic fermentation.

10. The method according to claim 1, wherein (i) comprises capturing CO₂ from a waste gas, or an industrial off-gas, optionally an off-gas from steel industry.

11. The method according to claim 1, wherein a CO containing waste gas is fed to the anaerobic fermentation, wherein CO is used as a carbon substrate and/or energy source for fermentation.

12. The method according to claim 11, wherein the CO containing waste gas further comprises CO₂, and wherein the waste gas is first fed to the anaerobic fermentation before capturing CO₂ from the waste gas.

13. The method according to claim 11, wherein the CO containing waste gas further comprises CO₂, and wherein the waste gas is first subjected to CO₂ capture before feeding to the anaerobic fermentation.

14. The method according to claim 1, wherein the reduced CO₂ product is a compound selected from carbon monoxide, methane, ethane, ethanol, ethylene, methanol, formaldehyde, acetaldehyde and 1-propanol; or an organic acid selected from formic acid, oxalic acid, glyoxylic acid, glycolic acid, acetic acid, tartaric acid, malonic acid and propionic acid; or a salt of said organic acid.

15. The method according to claim 14, wherein the reduced CO₂ product comprises formic acid, which is fed to the anaerobic fermentation, the aerobic fermentation, or both.

16. The method according to claim 14, wherein the reduced CO₂ product comprises CO, which is fed to the anaerobic fermentation.
